(19) United States
(12) Patent Application Publication
(10) Pub. No.: US 2010/0266104 A1
(43) Pub. Date: Oct. 21, 2010

(54) X-RAY STAND WITH VARIABLE JAW SIZE

(75) Inventor: Adrianus Van Der Ende, Son En Breugel (NL)

Correspondence Address: PHILIPS INTELLECTUAL PROPERTY & STANDARDS, P. O. Box 3001, BRIARCLIFF MANOR, NY, 10510, US

(73) Assignee: KONINKLIJKE PHILIPS ELETCRONICS N.V., Eindhoven (NL)

(21) Appl. No.: 12/063,776

(22) PCT Filed: Aug. 23, 2006
(86) PCT No.: PCT/IB06/52921

§ 371 Date: Feb. 14, 2008

(30) Foreign Application Priority Data
Aug. 31, 2005 (EP) 05107957.2

(51) Int. Cl. H05G 1/02; A61B 6/00

(52) U.S. Cl. 378/197

(57) ABSTRACT

An X-ray stand comprising a generally arcuate swing arm (10) on which is provided an X-ray tube (15) in a stationary housing (14) and an X-ray detector (16). The detector (16) is hingedly mounted via a smaller arm (20) to the swing arm (10), so as to enable the smaller arm (20) to pivot relative to the upper end of the swing arm (10). The detector plate (16) is mounted to the free end of the smaller arm (20) by means of a joint (22) which enables the detector plate (16) to be pivoted relative to the smaller arm (20). In addition, the X-ray tube (15) is mounted on a pivotal carriage (15a) within the housing (14) such that it can be relatively tilted, and the carriage (15a) in or on which the X-ray tube (15) is provided is mounted within the housing (14) on a sliding mechanism (30) which permits the selective rotational movement of the X-ray tube (15) within the housing (14). Thus, because the detector (16) and X-ray tube (15) can be moved in a substantially vertical direction relative to the patient (24) the maximum achievable jaw size (28) is increased without a significant increase in the size of the stand.

The invention relates generally to X-ray apparatus having a stand comprising a swing arm for supporting an X-ray tube and X-ray detector.

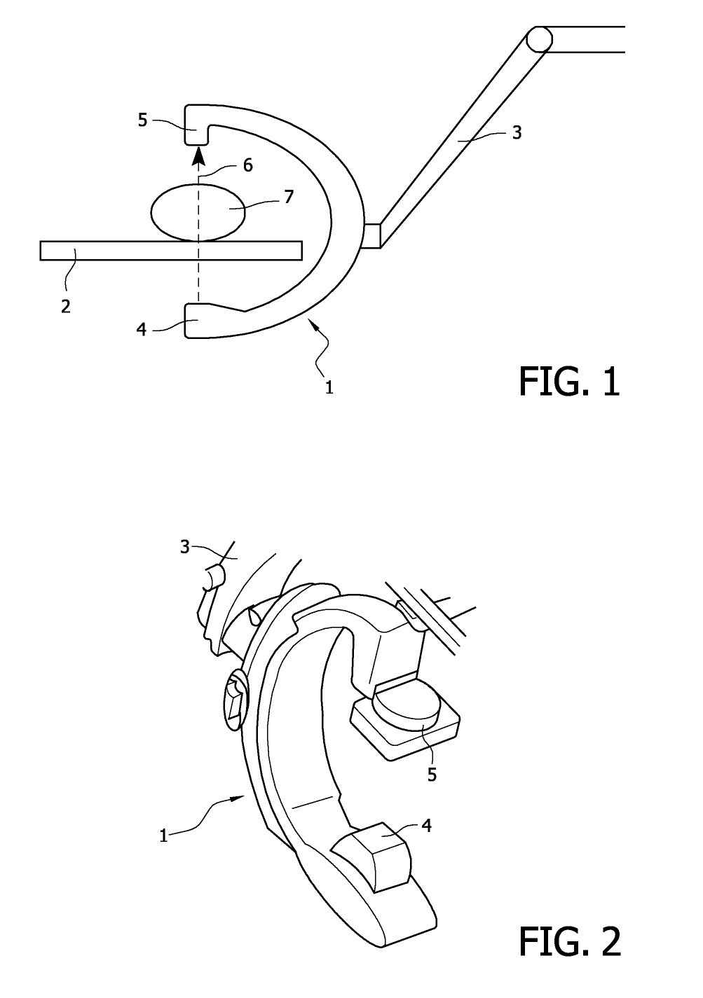

Referring to FIGS. 1 and 2 of the drawings, a typical X-ray system comprises a swing arm (C-arc or G-arc) 1 supported proximal a patient table 2 by an arm 3. Housed within the swing arm 1, there is provided an X-ray tube 4 and an X-ray detector 5, the X-ray detector 5 being arranged and configured to receive X-rays 6 which have passed through a patient 7 and generate an electric signal representative of the intensity distribution thereof. By moving the swing arm 1, the X-ray tube 4 and detector 5 can be placed at any desired location and orientation relative to the patient 7.

U.S. Pat. No. 4,775,994 describes a mobile X-ray examination unit including a C-arm carrying an X-ray tube and an X-ray detector, wherein the C-arm is hinged generally centrally so that the X-ray tube and detector are moveable relative to each other in a substantially vertical direction relative to the patient. This enables the jaw size of the C-arm to be varied according to the size of the patient.

It is highly desirable to minimise the size of the X-ray stand, both for convenience of use and storage. However, for certain investigations/interventions, the jaw size needs to be relatively very large, for example, 1050 mm and in order to achieve this jaw size in known devices, even in the arrangement described in U.S. Pat. No. 4,775,994 having a variable jaw size, the resultant X-ray stand is unacceptably large.

It is an object of the present invention to provide an X-ray stand having a C-arm with a variable jaw size, wherein the maximum achievable jaw size is increased relative to the prior art, without any significant increase in size of the stand.

In accordance with the present invention, there is provided an X-ray stand comprising a generally arcuate arm having opposing ends, wherein an X-ray source is provided at or adjacent one of said ends of said arm for generating an X-ray beam and an X-ray detector is provided at or adjacent the other of said ends of said arm, wherein said arcuate arm defines a jaw size comprising a distance between the primary axis of said X-ray beam and a point on said arm, wherein said X-ray stand further comprises means at or adjacent at least one of said ends of said arm for enabling movement of said detector and X-ray source relative to each other in a substantially radial direction relative to said arm, so as to selectively adjust said jaw size.

Thus, because the detector and/or X-ray tube can be moved in a substantially vertical direction relative to a respective end of the arm, the maximum achievable jaw size is significantly increased relative to the prior art, without a significant increase in the size of the stand.

Means may also be provided for enabling the orientation of the detector to be selectively adjusted relative to a patient positioned between the ends of said arcuate arm. In a preferred embodiment, the stand comprises an elongate mounting arm having a first end pivotally mounted at or adjacent a first end of said arcuate arm and an opposing second end to which said detector is coupled. Preferably, the detector is coupled to the second end of the mounting arm by means of a pivotal joint.

A sliding mechanism is beneficially provided for selectively moving said X-ray source in a substantially radial direction relative to the arcuate arm. For this purpose, the X-ray source may be mounted in or on a carriage coupled to a sliding mechanism. The X-ray source is beneficially mounted within a housing which is coupled to the arcuate arm, at or adjacent an end thereof, and is stationary relative thereto. In a preferred embodiment, means may be provided for enabling the orientation of the X-ray source to be selectively adjusted relative to a patient positioned between the ends of said arcuate arm.

These and other aspects of the present invention will be apparent from, and elucidated with reference to, the embodiment described herein.

An embodiment of the present invention will now be described by way of example only and with reference to the accompanying drawings, in which:

FIG. 1 is a schematic side view of an X-ray swing arm according to the prior art;

FIG. 2 is a schematic perspective view of an X-ray swing arm according to the prior art;

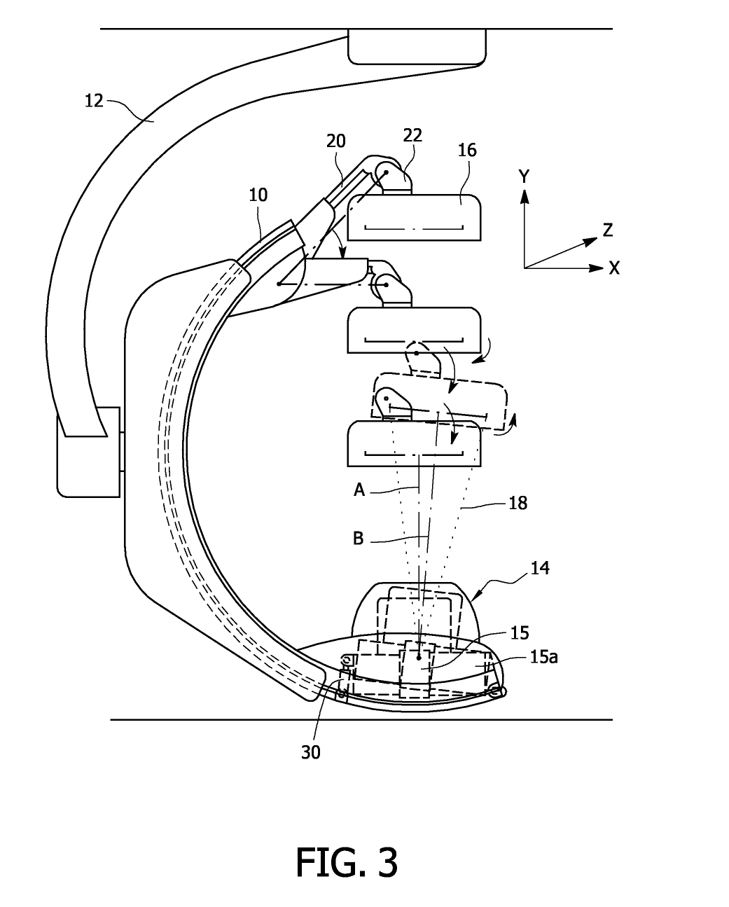

FIG. 3 is a schematic side view of an X-ray swing arm according to an exemplary embodiment of the present invention, in a number of different configurations;

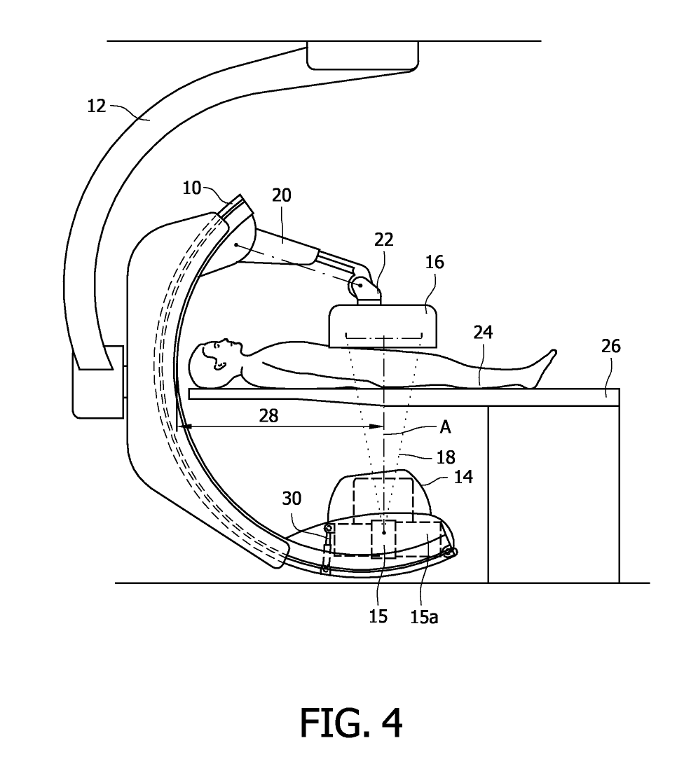

FIG. 4 is a schematic side view of an X-ray swing arm according to an exemplary embodiment of the present invention, when in use, defining a first jaw size; and

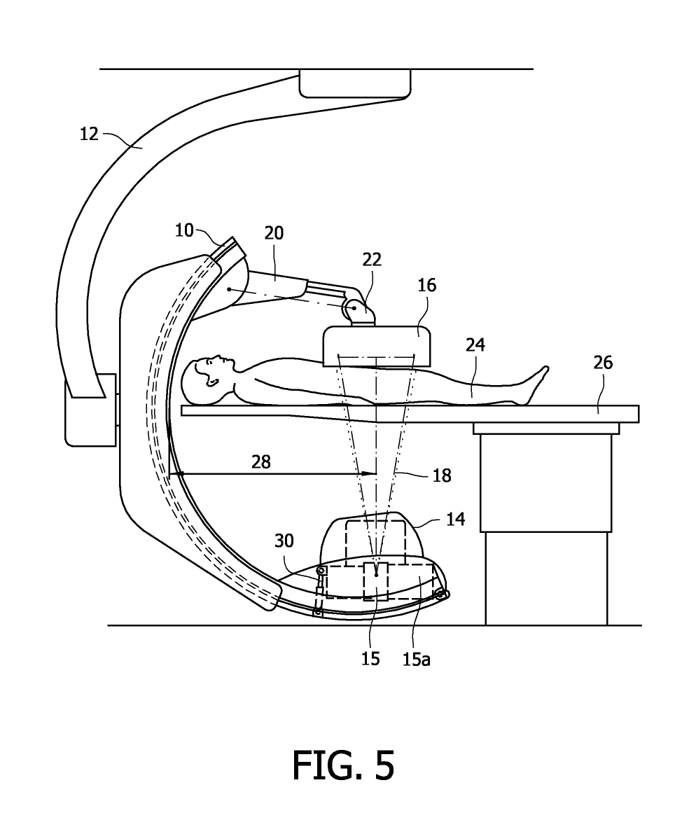

FIG. 5 is a schematic side view of an X-ray swing arm according to an exemplary embodiment of the present invention, when in use, defining a second jaw size.

Referring to FIG. 3 of the drawings, an X-ray stand according to an exemplary embodiment of the present invention comprises a generally arcuate swing arm (C-arc or G-arc) 10 supported by an arm 12. In this exemplary embodiment, the radius of the arcuate swing arm 10 may be, say, 940 mm. Mounted within the swing arm 10, there is provided an X-ray tube 15 in a housing 14 and an X-ray detector 16, the X-ray detector 16 being arranged and configured to receive X-rays 18 which have passed through a patient (not shown) and generate an electric signal representative of the intensity distribution thereof. By moving the swing arm 10, the X-ray tube 15 and detector 16 can be placed at any desired location and orientation relative to the patient.

The detector 16 is mounted via a smaller arm 20 to the swing arm 10, adjacent the upper end thereof. The smaller arm 20 is hingedly mounted to the swing arm 10 so as to enable the smaller arm 20 to pivot relative to the upper end of the swing arm 10, about a Z-axis. The detector plate 16 is mounted to the free end of the smaller arm 20 by means of a joint 22 which enables the detector plate 16 to be pivoted relative to the smaller arm 20, again about a Z-axis. Thus, an operator can adjust the position of the detector plate 16 in a substantially radial direction relative to the swing arm 10 by pivoting the smaller arm 20, and the operator can also adjust the orientation of the detector plate 16 relative to the free end of the smaller arm 20, so as to permit a number of different configurations, as shown in FIG. 3.

In addition, the X-ray tube 15 is mounted on a pivotal carriage 15a within the housing 14 such that it can be selectively tilted from a substantially horizontal orientation to an orientation at an angle to the X-axis, such that the primary axis of the X-ray beam 18 can be selectively changed from A to B.

Referring additionally to FIGS. 4 and 5, the carriage 15a in or on which the X-ray tube 15 is provided is mounted within the housing 14 on a sliding mechanism 30 which permits rotational movement of the X-ray tube 15 within the housing 14 (as indicated in FIG. 3). Thus, as shown in FIG. 4 of the drawings, in an exemplary configuration, the detector plate 16 is pivoted downwardly relative to the upper end of the swing arm 10 toward the patient 24 lying on the patient table 26 and the X-ray tube 15 is pivoted upwardly within the housing, toward the patient, as shown. As a result, a jaw size 28 of, say, 1030 mm can be achieved with a swing arm 10 of radius 940 mm. If, as shown in FIG. 5 of the drawings, a larger patient 24 is required to be accommodated, the configuration of the detector 16 and X-ray tube 15 may be adjusted such that a jaw size of, say 1050 mm can be achieved with a swing arm of radius 940 mm.

It should be noted that the above-mentioned embodiments illustrate rather than limit the invention, and that those skilled in the art will be capable of designing many alternative embodiments without departing from the scope of the invention as defined by the appended claims. In the claims, any reference signs placed in parentheses shall not be construed as limiting the claims. The word “comprising” and “comprises”, and the like, does not exclude the presence of elements or steps other than those listed in any claim or the specification as a whole. The singular reference of an element does not exclude the plural reference of such elements and vice-versa. The invention may be implemented by means of hardware comprising several distinct elements, and by means of a suitably programmed computer. In a device claim enumerating several means, several of these means may be embodied by one and the same item of hardware. The mere fact that certain measures are recited in mutually different dependent claims does not indicate that a combination of these measures cannot be used to advantage.

1. An X-ray stand comprising a generally arcuate arm having opposing ends, wherein an X-ray source is provided at or adjacent one of said ends of said arm for generating an X-ray beam and an X-ray detector is provided at or adjacent the other of said ends of said arm, wherein said arcuate arm defines a jaw size comprising a distance between a primary axis of said X-ray beam and a point on said arm, wherein said X-ray stand further comprises means at or adjacent at least one of said ends of said arm for enabling movement of said detector and X-ray source relative to each other in a substantially radial direction relative to said arm, so as to selectively adjust said jaw size.

2. The stand according to claim 1, further comprising means for enabling the orientation of the detector to be selectively adjusted relative to a patient positioned between the ends of said arcuate arm.

3. The stand according to claim 1, comprising an elongate mounting arm having a first end pivotally mounted at or adjacent a first end of said arcuate arm and an opposing second end to which said detector is coupled.

4. The stand according to claim 3, wherein said detector is coupled to the second end of the mounting arm by means of a pivotal joint.

5. The stand according to claim 1, wherein a sliding mechanism is provided for selectively moving said X-ray source in a substantially radial direction relative to the arcuate arm.

6. The stand according to claim 5, wherein the X-ray source is mounted in or on a carriage coupled to said sliding mechanism.

7. The stand according to claim 1, further comprising means for enabling the orientation of the X-ray source to be selectively adjusted relative to a patient positioned between the ends of said arcuate arm.

8. The stand according to claim 1, wherein said X-ray source is mounted within a housing which is coupled to the arcuate arm, at or adjacent an end thereof, and is stationary relative thereto.

9. An x-ray imaging system comprising:
an x-ray source mounted at a first end of an x-ray arm;
an x-ray detector mounted at a second end of the x-ray arm; and
means for adjusting a distance between a primary axis of an x-ray beam and a point on the arm.

10. The system of claim 9 further comprising means for selectively moving the x-ray source in a substantially radial direction relative to the arm.

11. An x-ray imaging system comprising:
an x-ray source that generates an x-ray beam;
an x-ray detector that detects the x-ray beam from the x-ray source; and
an arm to which the x-ray source and x-ray detector are attached;
wherein the system is operable in a first configuration in which the distance between a point on the arm and the x-ray beam is a first distance; and
wherein the system is operated in a second configuration in which the distance between the position on the arm and the x-ray beam is a second distance; the first and second distances being different.
